(19) United States
(12) Patent Application Publication
(10) Pub. No.: US 2013/0123498 A1
(43) Pub. Date: May 16, 2013

(54) PROCESS FOR THE PREPARATION OF ETRAVIRINE AND INTERMEDIATES IN THE SYNTHESIS THEREOF

(71) Applicants: Maja Sepelj MAJER, Zagreb (HR); Irena KRIZMANIC, Sisak (HR); Gordana VRBANEC, Zagreb (HR)

(72) Inventors: Maja Sepelj MAJER, Zagreb (HR); Irena KRIZMANIC, Sisak (HR); Gordana VRBANEC, Zagreb (HR)

(21) Appl. No.: 13/655,750

(22) Filed: Oct. 19, 2012

Related U.S. Application Data
(60) Provisional application No. 61/548,952, filed on Oct. 19, 2011.
(60) Provisional application No. 61/562,174, filed on Nov. 21, 2011.
(60) Provisional application No. 61/651,286, filed on May 24, 2012.

(51) Int. Cl. C07D 239/48

(52) U.S. Cl. CPC C07D 239/48
USPC 544/321; 544/323

(57) ABSTRACT

The present invention includes compounds of Formula 1 of the following structure:

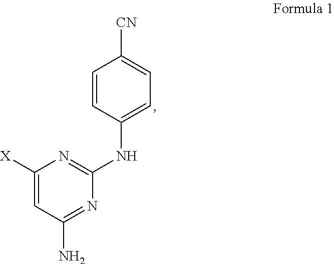

or salts thereof, wherein X is a halogen or a tosyl group. Processes for preparing the compounds of Formula 1 are disclosed in the patent application. The compounds of Formula 1 are useful as intermediates for preparation of Etravirine. The present invention is also directed toward processes for preparing Etravirine.

### CROSS REFERENCE TO RELATED APPLICATIONS

This patent application claims the benefits of U.S. Provisional Patent Application Nos. 61/548,952, 61/562,174 and 61/651,286 filed on Oct. 19, 2011, Nov.21, 2011, and May 24, 2012, respectively, the disclosures of which are incorporated by reference.

### FIELD OF THE INVENTION

The present invention relates to processes for preparing Etravirine, to novel intermediates useful in those processes, and to other intermediates useful in the synthesis of Etravirine.

### BACKGROUND OF THE INVENTION

Etravirine (“ETV”), 4-(6-amino-5-bromo-2-(4-cyanophenylamino)pyrimidin-4-yloxy)-3,5-dimethylbenzonitrile, having the following chemical structure:

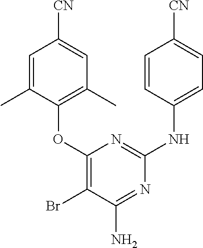 [SMILES: Cc1cc(C#N)cc(C)c1Oc1nc(Nc2ccc(C#N)cc2)nc(N)c1Br]

is a drug used for the treatment of HIV.

Etravirine was formerly known as TMC-125, and has the brand name INTELENCE®.

Etravirine, certain of its analogs and their syntheses are described in U.S. Pat. No. 7,037,917. In this patent, Etravirine is prepared by reacting a tetra halogenated pyrimidine derivative with an aminobenzene derivative, and optionally brominating the product of that reaction, to obtain Etravirine. This process includes two steps that require chromatographic purification of intermediates, and the final product is obtained in very poor overall yield. EP 945443 describes a general process for the preparation of anilinopyrimidine derivatives, and salts thereof, e.g., by aminolysis reactions of pyrimidine compounds containing leaving groups.

Bioorg.Med.Chem.Lett. 11 (2001) 2235-2239 describes a process for preparing Etravirine, according to the following scheme:

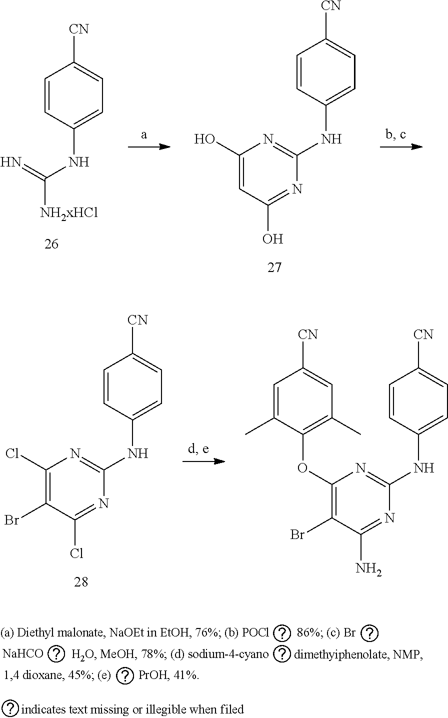

wherein Etravirine is obtained in a very poor overall yield of less than 10%.

WO 01/85700 describes a preparation of Etravirine analogues which do not include an NH₂ group. In this process, the Etravirine analogues are purified by column chromatography and are obtained in very poor overall yield of about 2-5%.

WO 2006/094930, WO 2008/071587 and WO 2000/027825 describe different processes for preparing different Etravirine intermediates and derivatives, all of which require at least one chromatographic purification step.

WO 2008/068299 discloses that Etravirine has very poor solubility in water, and describes a hydrobromide salt of Etravirine, which is prepared by dissolving Etravirine in dichloromethane and reacting with hydrobromic acid.

WO2011/017079 and WO2010/150279 disclose additional processes for preparing Etravirine and Etravirine intermediates.

The disclosures of the patents, patent applications, and publications cited in this application are hereby incorporated herein by reference in their entirety.

The present invention provides a novel process for preparing Etravirine.

### SUMMARY OF THE INVENTION

The present invention provides novel intermediates of Etravirine, a process for preparing the novel intermediates and the use of the novel intermediates in the preparation of Etravirine and other Etravirine intermediates. Also provided are processes for the synthesis of Etravirine, synthesis of the novel Etravirine intermediates, and synthesis of other Etravirine intermediates as described below.

### BRIEF DESCRIPTION OF THE FIGURES

FIG. 1 provides a full-width ¹H NMR spectrum of 4-(4-amino-5-bromo-6-chloro-pyrimidin-2-ylamino)benzonitrile (“Compound 3a”).

FIG. 2 provides a ¹H NMR spectrum of 4-(4-amino-5-bromo-6-chloropyrimidin-2-ylamino)benzonitrile (“Compound 3a”), in the range of 10.4-6.5 ppm.

FIG. 3 provides a full-width ¹³C NMR spectrum of 4-(4-amino-5-bromo-6-chloro-pyrimidin-2-ylamino)benzonitrile (“Compound 3a”).

FIG. 4 provides a ¹³C NMR spectrum of 4-(4-amino-5-bromo-6-chloropyrimidin-2-ylamino)benzonitrile (“Compound 3a”), in the range of 164-86 ppm.

FIG. 5 provides a full-width ¹H NMR spectrum of 4-(4-amino-6-chloropyrimidin-2 ylamino)benzonitrile (“Compound 1 a”).

FIG. 6 provides a ¹H NMR spectrum of 4-(4-amino-6-chloropyrimidin-2 ylamino)-benzonitrile (“Compound 1a”), in the range of 10.2-5.8 ppm.

FIG. 7 provides a full-width ¹³C NMR spectrum of 4-(4-amino-6-chloropyrimidin-2 ylamino)benzonitrile (“Compound 1a”).

FIG. 8 provides a¹³C NMR spectrum of 4-(4-amino-6-chloropyrimidin-2 ylamino)-benzonitrile (“Compound 1a”), in the range of 170-90 ppm.

### DETAILED DESCRIPTION OF THE INVENTION

The present invention relates to novel intermediates and process for preparing Etravirine, and to synthetic intermediates in that process.

The process of the present invention has less steps, is more efficient and less costly than the known processes. For example, the process of the present invention comprises only 3 steps and provides Etravirine in higher overall yield and purity without the need to invest efforts in purification. For example, it can be purified by forming an Etravirine salt, and the product is achieved with good quality. For these reasons, this process is of importance to manufacturing Etravirine on an industrial scale.

As used herein, the term “room temperature” refers to a temperature from about 20° C. to about 30° C. Usually, room temperature ranges from about 20° C. to about 25° C.

As used herein, the term “overnight” refers to a period of between about 15 hours and about 20 hours, typically between about 16 hours to about 20 hours.

As used herein, unless indicated otherwise, the term “isolated” refers to Etravirine and Etravirine intermediates, such as 4-(4-amino-6-substituted pyrimidin-2-ylamino)benzonitrile (Compounds of Formula 1, or simply Compound 1) and in particular 4-(4-amino-6-chloropyrimidin-2 ylamino)benzonitrile (referred to as Compound 1a), that are physically separated from the reaction mixtures in which they were formed.

The present invention provides a novel process for preparing Etravirine or a salt thereof via the novel intermediates: 4-(4-amino-6-substituted pyrimidin-2-ylamino)benzonitrile (referred to as Compounds of Formula 1) and salts thereof of the following structure:

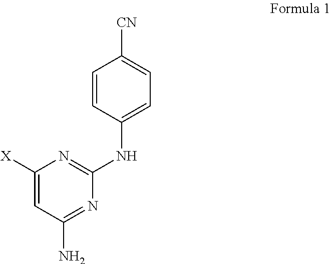

wherein X is a halogen, e.g., —Br, —Cl, —I or —F, or a tosyl group.

When X is —Cl, the compound is 4-(4-amino-6-chloropyrimidin-2 ylamino)benzonitrile (Compound 1a) of the following structure:

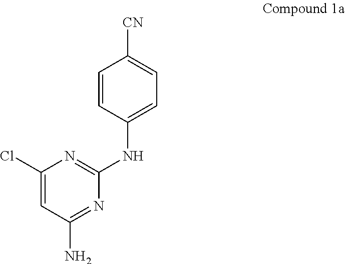 [SMILES: N#Cc1ccc(Nc2nc(N)cc(Cl)n2)cc1]

Salts of the Compounds of Formula 1, Formula 2 and Formula 3 can be inorganic salts, such as HCl, HBr, HI or HClO₄ salts or organic salts, such as tosylate, acetate, mesylate, nitrate, phosphate, benzenesulfonate, ethanesulfonate or aminobenzenesulfonate.

The process of the present invention can be illustrated by scheme 1, below.

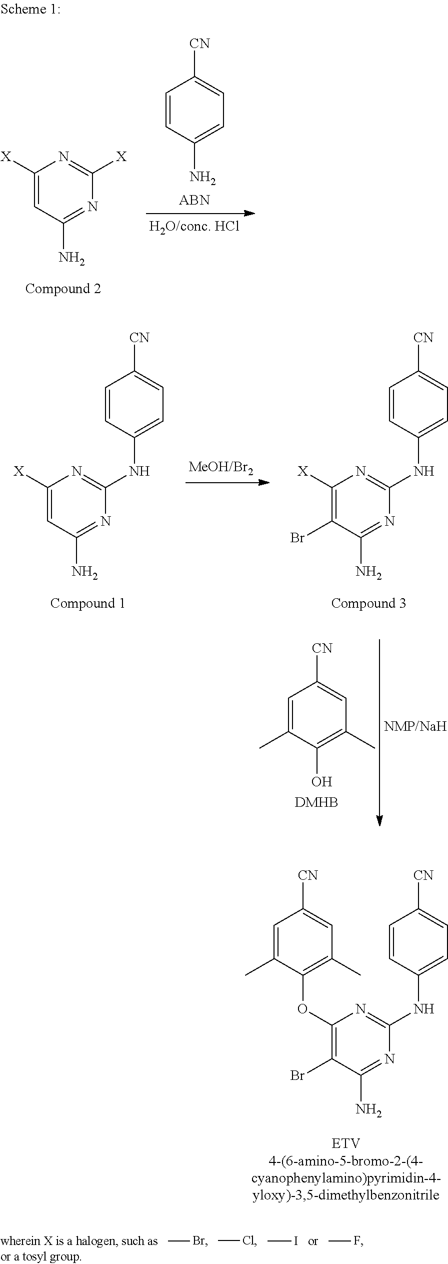

In one of the embodiments, the present invention encompasses Compounds of Formula 1 and salts thereof, for example Compound 1a.

The Etravirine intermediate, Compound 1, for example, Compound la, can be isolated, and, according to some embodiments, this intermediate is crystalline.

The above described Compounds of Formula 1, e.g., Compound 1a, can be used to prepare Etravirine, Etravirine intermediates and salts thereof.

In another embodiment, the present invention provides a process for preparing the above Compounds of Formula 1, e.g., Compound la and salts thereof. The process comprises reacting a Compound of Formula 2 (alternatively referred to herein simply as Compound 2) such as a 2,6-di-substituted pyrimidin-4-amine, or a salt thereof of the following structure:

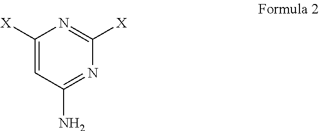

with 4-aminobenzonitrile (“ABN”) of the following structure:

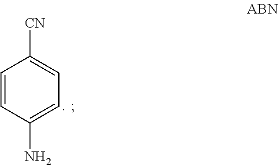

wherein X is a halogen, e.g., —Br, —Cl, —I or —F, or a tosyl group

When X is —Cl, the process comprises reacting 2,6-dichloropyrimidin-4-amine (referred to as Compound 2a), or a salt thereof, of the following structure:

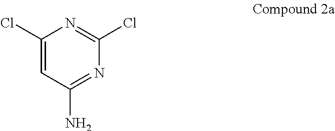

### and ABN.

Salts of the Compound of Formula 2, in particular Compound 2a, can be as described above.

The process for preparing a Compound of Formula 1, e.g., Compound 1a, can be done, for example, under the conditions specified hereinafter.

When a salt of compound 1 is obtained, it can then be basified to obtain Compound 1 in a free base form or, alternatively, the salt can be used in the next step directly.

The above process can further comprise converting the obtained Compound of Formula 1, e.g., Compound 1a, to Etravirine or an Etravirine salt.

The present invention provides a process for preparing Etravirine and salts thereof comprising preparing one or more of the Compounds of Formula 1, e.g., Compound 1a, or salts thereof, by the process of the present invention, and converting said one or more of the Compounds of Formula I or salts thereof to Etravirine or a salt thereof. The conversion can be done, for example, by brominating the Compound of Formula 1 to obtain 4-(4-amino-5-bromo-6-substituted pyrimidin-2-ylamino)benzonitrile (referred to as a Compound of Formula 3 or simply as Compound 3), and reacting the obtained Compound 3 and 4-hydroxy-3,5-dimethylbenzonitrile (“DMHB”) to obtain Etravirine. When X is —Cl, i.e., Compound 1a, this intermediate can be brominated to obtain 4-(4-amino-5-bromo-6-chloropyrimidin-2-ylamino)benzonitrile (referred to as Compound 3a), which can then be reacted with DMHB to obtain Etravirine.

The process can further include converting Etravirine to an Etravirine salt, for example by reacting Etravirine with a suitable acid.

In another embodiment, the present invention encompasses a process for preparing a 4-(4-amino-5-bromo-6-substituted pyrimidin-2-ylamino)benzonitrile (referred to as a Compound of Formula 3, or simply as Compound 3), or a salt thereof of the following structure:

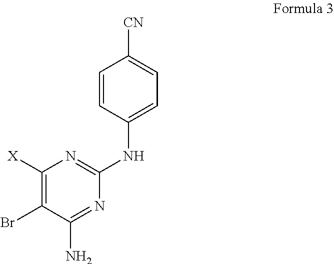

wherein X is a halogen, e.g., —Br, —Cl, —I or —F, or a tosyl group; comprising preparing a Compound of Formula 1 or a salt thereof by the process of the present invention, and converting it to a Compound of Formula 3 or a salt thereof. The conversion can be done, for example, by brominating the Compound of Formula 1.
When X is —Cl, i.e., Compound 1a, the compound is brominated to obtain Compound 3a of the following structure:

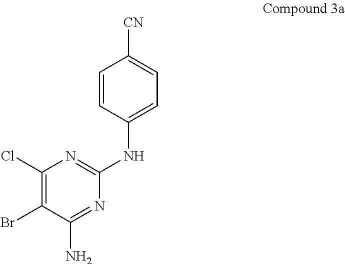 [SMILES: N#Cc1ccc(Nc2nc(N)c(Br)c(Cl)n2)cc1]

Salts of a Compound of formula 3, in particular Compound 3a, can be as described above.

The process for preparing a Compound of Formula 3, such as Compound 3a, can be done, for example, under the conditions specified hereinafter.

The above process can further comprise converting the obtained Compound of Formula 3 or a salt thereof, e.g., Compound 3a to Etravirine or an Etravirine salt. The conversion of the Compound of Formula 3, e.g., Compound 3a to Etravirine can be done by reacting the compound and 4-hydroxy-3,5-dimethylbenzonitrile (“DMHB”) of the following structure:

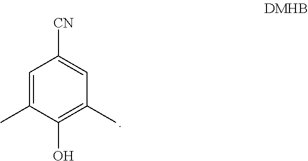

When a salt of the Compound of Formula 3 is used, the obtained Etravirine salt can be basified to obtain Etravirine in a free base form. Optionally, prior to the basification a re-crystallization and/or conversion to a second Etravirine salt, such as a tosylate, nitrate, phosphate, benzenesulfonate, ethanesulfonate or aminobenzenesulfonate salt; can be performed.

In yet another embodiment the present invention encompasses a process for preparing Etravirine comprising:
a) Reacting a Compound of Formula 2 such as a 2,6-di-substituted pyrimidin-4-amine and 4-aminobenzonitrile (“ABN”) to obtain a Compound of Formula 1 such as a 4-(4-amino-6-substituted pyrimidin-2-ylamino)benzonitrile;
b) Brominating the Compound of Formula 1 to obtain a Compound of Formula 3 such as a 4-(4-amino-5-bromo-6-substituted pyrimidin-2-ylamino)benzonitrile; and
c) Reacting the Compound of Formula 3 and 4-hydroxy-3,5-dimethylbenzonitrile (“DMHB”) to obtain Etravirine or a salt thereof; as shown in the following scheme:

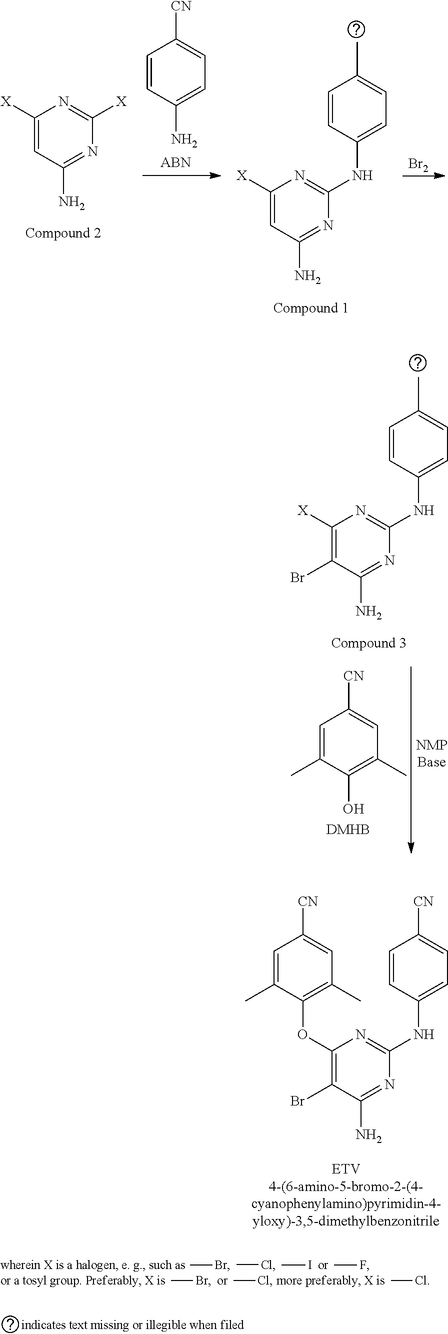

When X is —Cl, the Compound 2a can be reacted with ABN in step a) to obtain Compound 1a, which can be brominated in step b) to obtain Compound 3a; which can be reacted with DMHB in step c) to obtain Etravirine.

The reaction in step a) can be done in the presence of an aqueous solvent, such as water, or a mixture of water and a polar aprotic organic solvent, such as N-methyl-2-pyrrolidone (“NMP”), dimethylacetamide (“DMA”), dimethyl sulfoxide (“DMSO”), dioxane, or acetonitrile; an alcohol solvent, for example a C₁-C₄ alcohol, such as methanol, ethanol, 1-propanol, 2-propanol, n-butanol, 2-butanol, isobutanol or tent-butanol; a ketone solvent, such as acetone or methyl ethyl ketone (“MEK”); or any mixture thereof. This reaction can optionally be performed in the presence of an acid. The acid can be an organic acid such as acetic acid, trichloroacetic or a sulfonic acid (eg MsOH or TsOH); or an inorganic acid, such as hydrobromic acid, hydrochloric acid H₂SO₄, HNO₃, H₃PO₄. When a salt of the starting Compound of Formula 2, and/or an excess of acid are used, typically a salt of the Formula 1 compound is obtained, which can then be basified to obtain the Compound of Formula 1 in a free base form.

Typically, the reaction in step a) is done at a suitable temperature, for example, from about 60° C. to about 130° C., or from about 90° C. to about 110° C. The reaction can be continued for a period of time sufficient to complete the reaction, for instance from about 4 hours to about 24 hours, or from about 5 hours to about 6 hours.

The bromination in step b) is typically done in the in the presence of at least one suitable solvent. Suitable solvents include alcohol solvents, particularly C₁-C₄ alcohol solvents, such as methanol, ethanol, 1-propanol, 2-propanol, 1-butanol or 2-butanol; organic acids, such as C₁-C₄ organic acids, e.g., acetic acid; amide solvents, such as dimethylformamide (DMF) or dimethylacetamide (DMA), ether solvents, such as tetrahydrofuran (THF), nitrile solvents, such as acetonitrile, and mixtures thereof. The bromination can be performed at a suitable temperature, for example, from about 0° C. to about 30° C., from about 10° C. to about 30° C., from about 15° C. to about 30° C., from about 5° C. to about 15° C., or from about 20° C. to about 25° C. The reaction can be continued for a period of time sufficient to complete the reaction, for instance from about 1 hour to about 4 hours, from about 2 hour to about 4 hours, or from about 1 hour to about 3 hours. In some instances, the reaction can be completed in about 1 to 2 hours.

Step c) can be done in the presence of at least one suitable solvent and at least one suitable base. Suitable solvents include, for example, ether solvents, such as THF; halogenated hydrocarbons, such as dichloromethane or chloroform; and polar aprotic organic solvents, such as NMP, DMF, DMA, hexamethylphosphoramide (“HMPT”) acetonitrile, DMSO and mixtures thereof. In some preferred embodiments, the solvent is NMP.

Suitable bases include inorganic bases, such as metal hydroxides, e.g., sodium hydroxide and potassium hydroxide; metal hydrides, such as sodium hydride and calcium hydride and carbonate bases, such as potassium carbonate and sodium carbonate, and mixtures of these bases.

The above reaction of step c) can be carried out at a suitable temperature, for example, from about 110° C. to about 150° C., or from about 115° C. to 130° C. The reaction can be continued for a period of time sufficient to complete the reaction, for example from about 6 hours to about 25 hours or from about 6 hours to about 12 hours.

The obtained Etravirine can be purified by any suitable method known in the art, for example, by crystallization. Alternatively, Etravirine can be purified by converting it to an Etravirine salt, which can then be recrystallized. Such a conversion is typically done by reacting the Etravirine with an appropriate acid to obtain the corresponding salt.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

Nuclear Magnetic Resonance (“NMR”) method (¹H-NMR, ¹³C-NMR, ¹⁵N-NMR and ⁸¹Br-NMR) The sample was dissolved in DMSO-d₆. A small amount of TMS was added as a reference. NMR spectra were acquired on a Varian Unity Inova 300 MHz spectrometer equipped with ID and ATB probes at the Slovenian NMR Centre at the National Institute of Chemistry in Ljubljana. Sample temperature was set at 298 K ¹H and ¹³C NMR chemical shifts were referenced with respect to TMS (δ 0.0 ppm).

### EXAMPLES

### Example 1a

### Preparation of 4-(4-amino-6-chloropyrimidin-2-ylamino)benzonitrile (“Compound 1 a”)

To a suspension of 4-aminobenzonitrile (ABN; 0.72 g; 6.1 mmol) in water (100 ml), was added 2,6-dichloropyrimidin-4-amine (“DCAP”; 1.0 g; 6.10 mmol) at room temperature. The resulting suspension was heated at 100° C. and stirred for 18 hours. Hydrochloric acid (12M, 3.1 ml; 36.59 mmol; 6 eq) was added, and the resulting mixture was stirred for 4.5 hours. Additional hydrochloric acid added (12M, 1.55 ml; 18.3 mmol; 3 eq), and this mixture was stirred for 5 hours at 100° C., and for an additional 65 hours at room temperature. The suspension was then cooled to 0-5° C. and the pH was adjusted with 10% NaOH solution to 13.8, and the mixture was then stirred 1 hour at 0-5° C. A solid was separated from the suspension by filtration and washed with water (25 ml) and dried (4 h/40° C./10 mbar).
Yield: 1.28 g (85.4%) of 4-(4-amino-6-chloropyrimidin-2-ylamino)benzonitrile.
Purity (HPLC/MS): 94.4 Area %
M+246.4 (±2)

¹H NMR (DMSO-d₆, δ): 9.86 (s, 1H, NH), 7.95 (d, 2H, Ph-b, J₁=8.8 Hz), 7.66 (d, 2H, Ph-c, J₁=8.8 Hz), 7.13 (br, 2H, NH₂), 6.05 (s, 1H, H5)

¹³C NMR (DMSO-d₆, δ): 165.02 (s, C2), 158.8 (s, C6), 157.6 (s, C4), 144.9 (s, Ph-a), 132.8 (d, Ph-c), 119.5 (s, CN), 118.3 (d, Ph-b), 102.2 (s, Ph-d), 95.3 (s, C5)

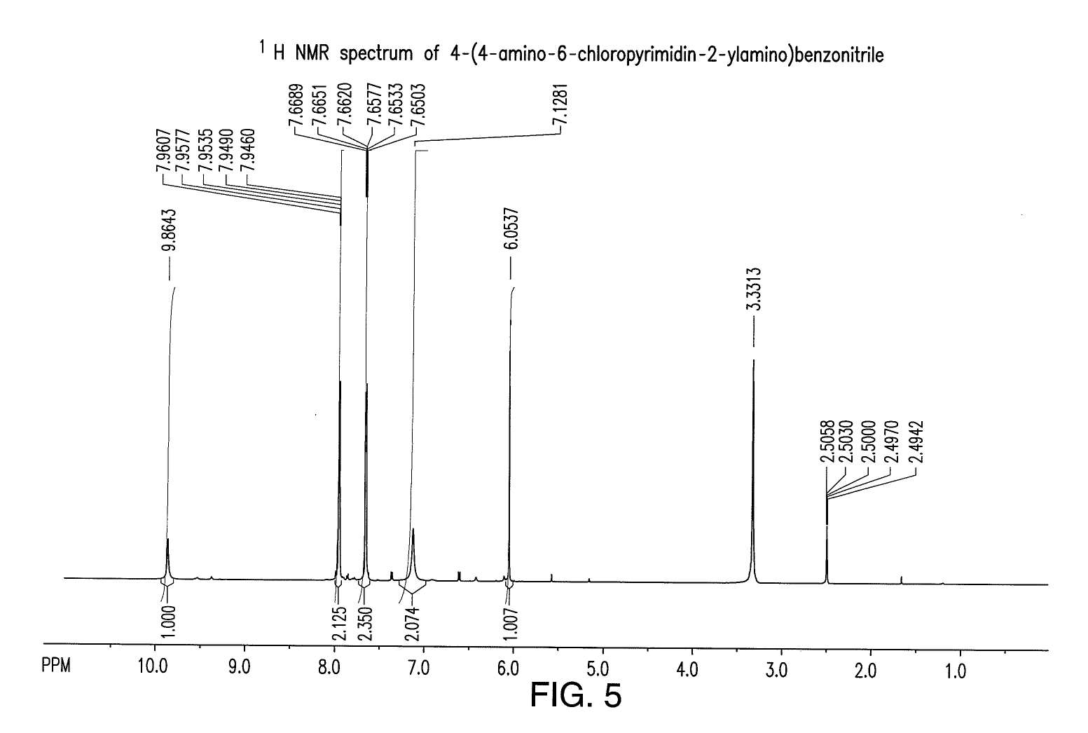

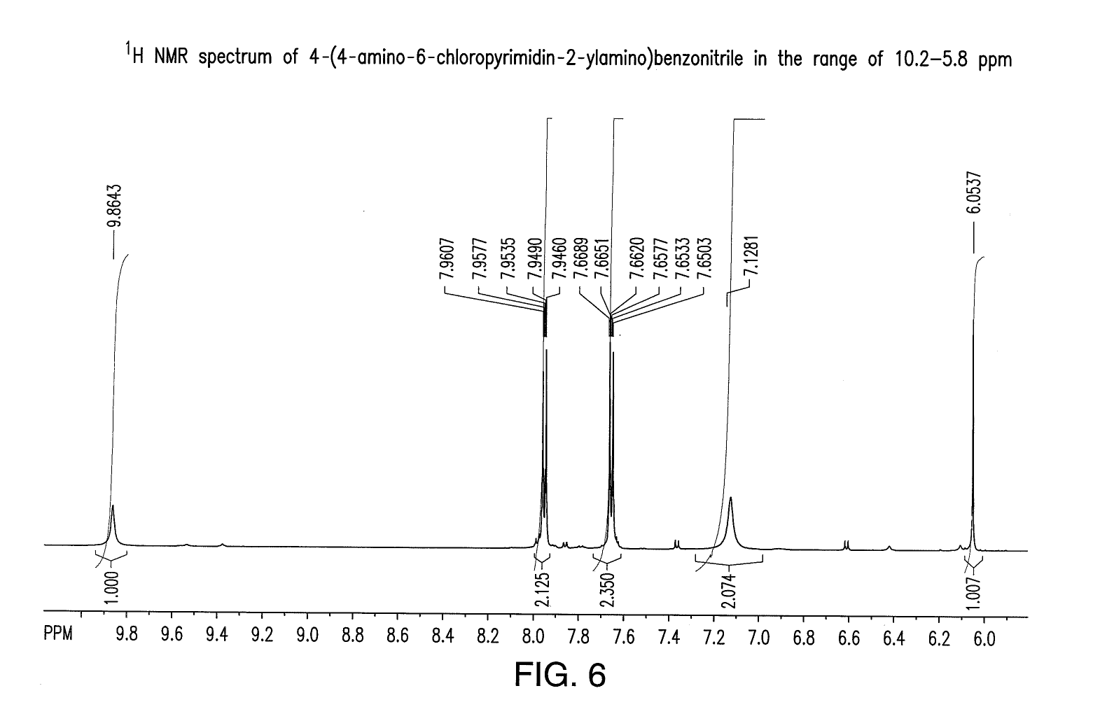

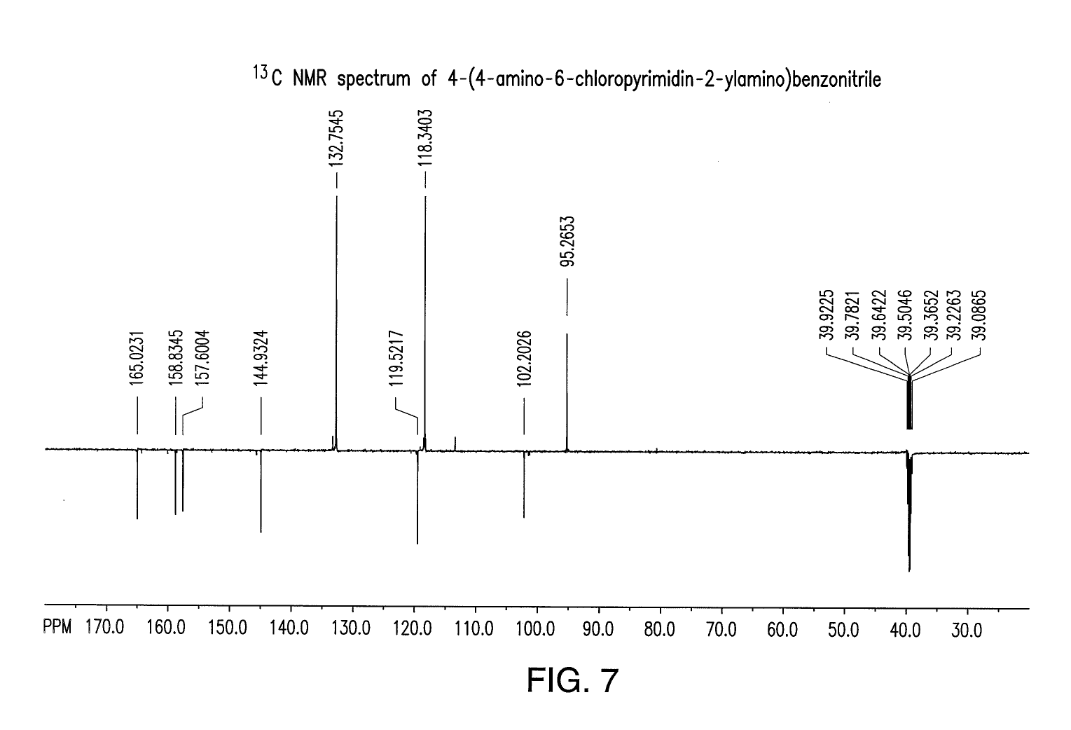

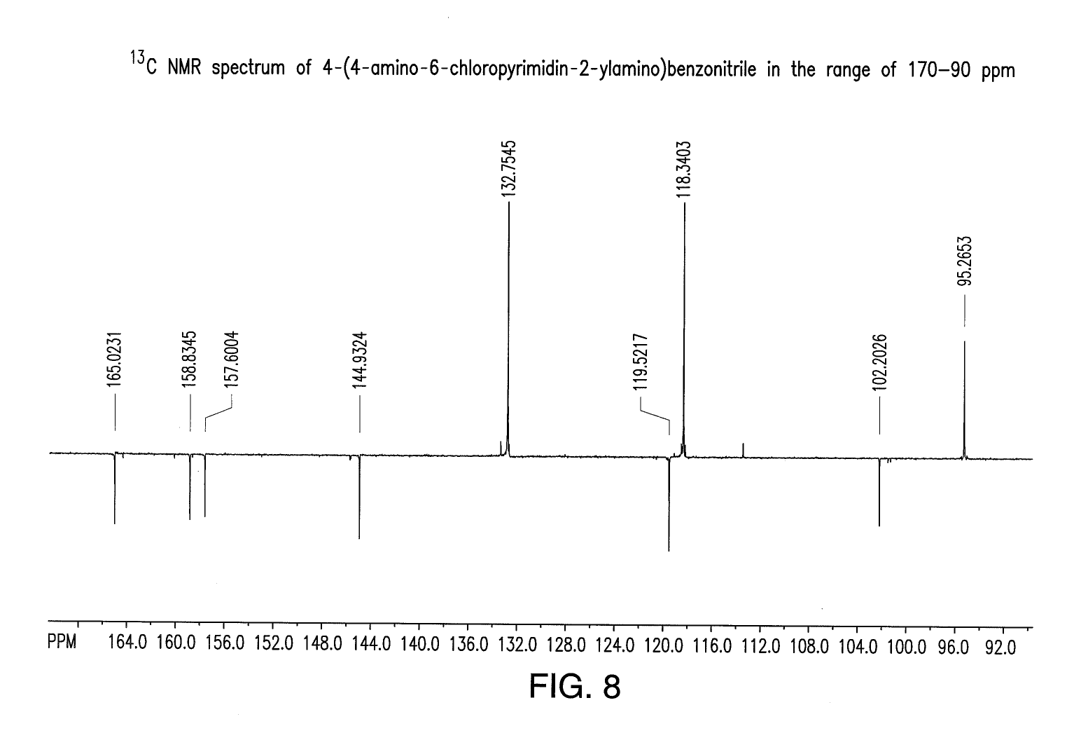

¹H NMR and ¹³C NMR spectra are shown in FIGS. 5-6 and 7-8, respectively.

### Example 1b

### Preparation of 4-(4-amino-6-chloropyrimidin-2-ylamino)benzonitrile (“Compound 1a”)

To the suspension of 2,6-dichloropyrimidin-4-amine (DCAP, 16.7 g, 101.8 mmol) and 4-aminobenzonitrile (ABN, 10 g, 84.7 mmol) in water (420 ml), concentrated hydrochloric acid (25.8 ml, 304.3 mmol) was added. The reaction mixture was heated to 100° C. and stirred for 2 h. Additional hydrochloric acid (25.8 ml, 304.3 mmol) was added, and the reaction mixture was stirred for another 2 h. A second portion of DCAP (5.57 g, 33.9 mmol) was added, and the stirring was continued for an additional hour. The reaction mixture was then cooled to 0-5° C., and left stirring over 1 h. The obtained suspension was filtered and the collected solid crude product was washed with water (200 ml) and dried (15 h/65° C./10 mbar).

Yield: 20.2 g (85%) of 4-(4-amino-6-chloropyrimidin-2-ylamino)benzonitrile, hydrochloride salt

Purity (HPLC/MS): 74 Area %.

M+- HCl=246

The 4-(4-amino-6-chloropyrimidin-2-ylamino)benzonitrile, hydrochloride salt (20.2 g, 71.6 mmol) was ground using mortar and pestle and suspended in acetone (50 ml). Aqueous sodium hydroxide (10%, 36.7 g, 91.9 mmol) was added dropwise over 5 min. The resulting suspension was heated at 40° C. for 40 min. Water (100 ml) was added dropwise over 25 min, and the reaction mixture was stirred for an additional 15 min at 40° C. The obtained suspension was cooled to room temperature over 40 min and then stirred at 0-5° C. for 1 h. The crude product was filtered off and washed with water (50 ml) and dried (15 h/65° C./10 mbar).

Yield: 15.8 g (90%) of 4-(4-amino-6-chloropyrimidin-2-ylamino)benzonitrile

Purity (HPLC/MS): 78 Area %.

M+246

Total yield: 76.5%

### Example 2a

### Preparation of 4-(4-amino-5-bromo-6-chloropyrimidin-2-ylamino)benzonitrile (“Compound 3a”)

A suspension of 4-(4-amino-6-chloropyrimidin-2-ylamino)benzonitrile (“CAPBN”, compound 1a; 0.4 g; 1.628 mmol) in methanol (5 ml) was cooled to 0-5° C. Bromine (167.2 μl; 3.26 mmol; 2 eq) was added dropwise to the suspension over 10 minutes. The resulting reaction mixture was stirred at 0-5° C. for 2 hours, and then water (0.83 ml) and 10% aqueous NaOH (1.95 ml; 4.88 mmol; 3 eq) was added dropwise. The resulting suspension was stirred 1 hour at 0-5° C. A solid was separated from the suspension by filtration and washed with a MeOH/water mixture (1:1; 3 ml), and dried (4 h/40° C./10 mbar).
Yield: 423 mg (80.1%) of 4-(4-amino-5-bromo-6-chloropyrimidin-2-ylamino)benzonitrile.
Purity (HPLC/MS): 95.71 Area %
M+326.4 (±2)

¹H NMR (DMSO-d₆, δ): 10.06 (s, 1H, NH), 7.96 (d, 2H, Ph-b, J₁=8.8 Hz), 7.70 (d, 2H, Ph-c, J₁=8.8 Hz), 7.50-6.50 (br, 2H, NH₂)

¹³C NMR (DMSO-d₆, δ): 161.9 (s, C2), 157.0 (s, C6), 156.7 (s, C4), 144.7 (s, Ph-a), 132.9 (d, Ph-c), 119.6 (s, CN), 118.5 (d, Ph-b), 102.6 (s, Ph-d), 89.8 (s, C5)

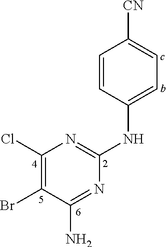 [SMILES: N#Cc1ccc(Nc2nc(N)c(Br)c(Cl)n2)cc1]

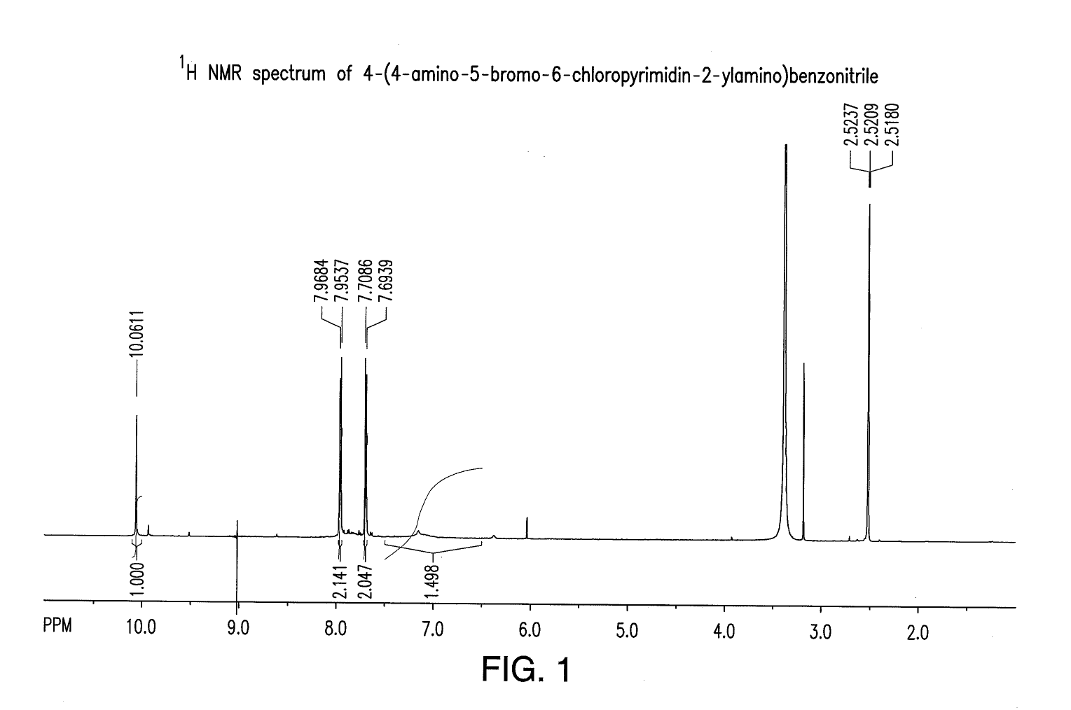

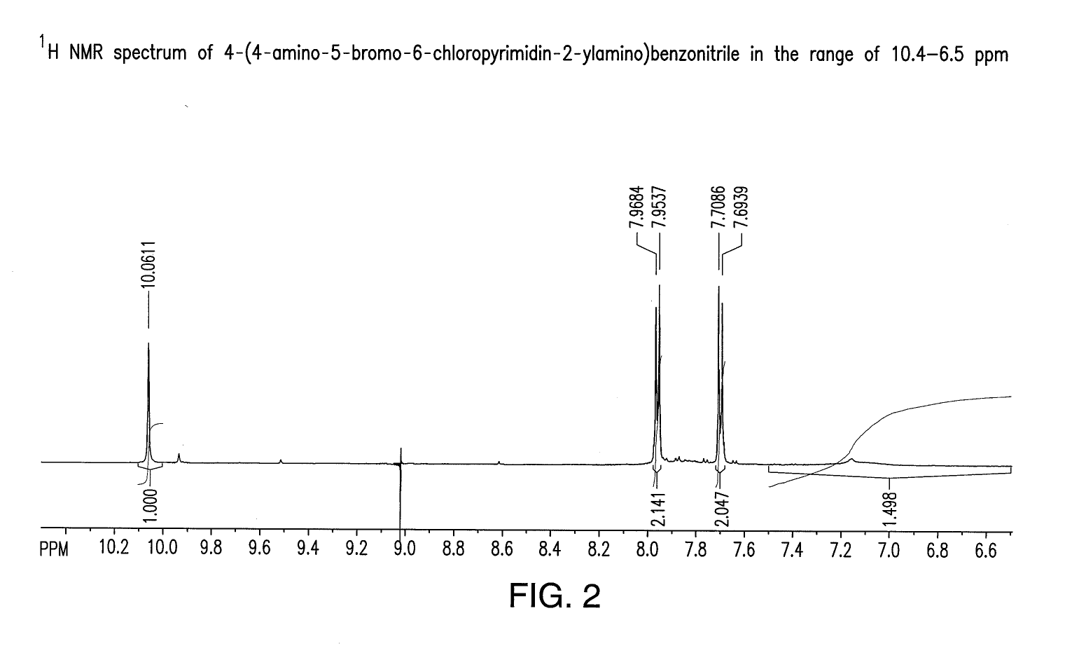

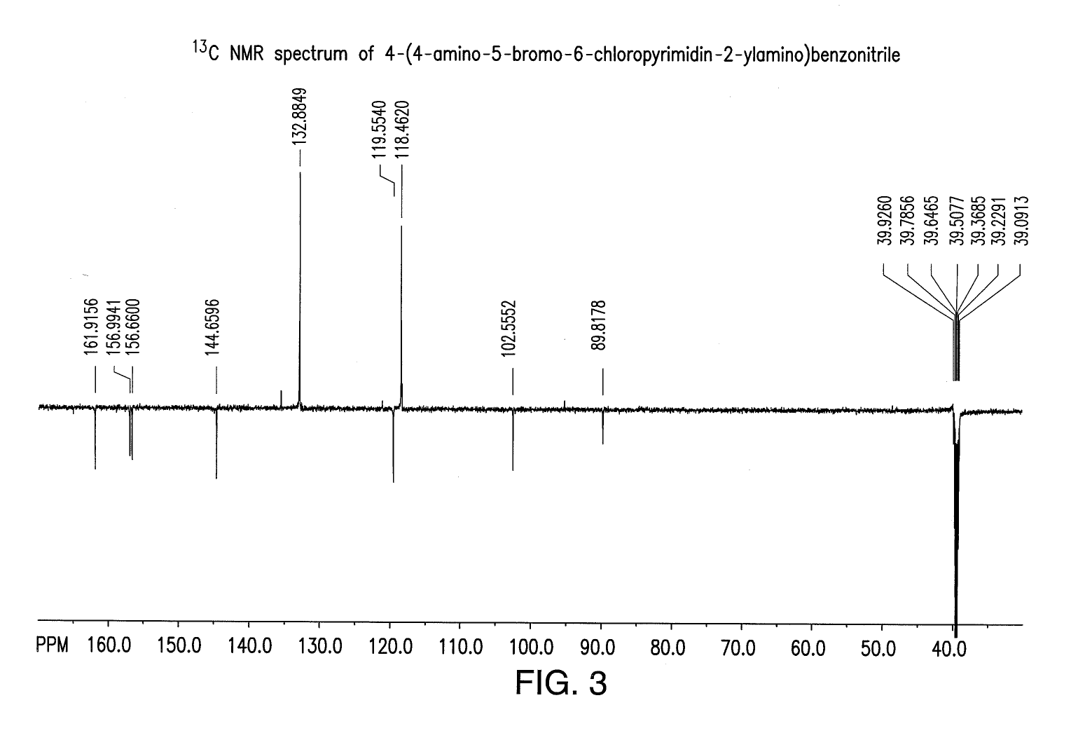

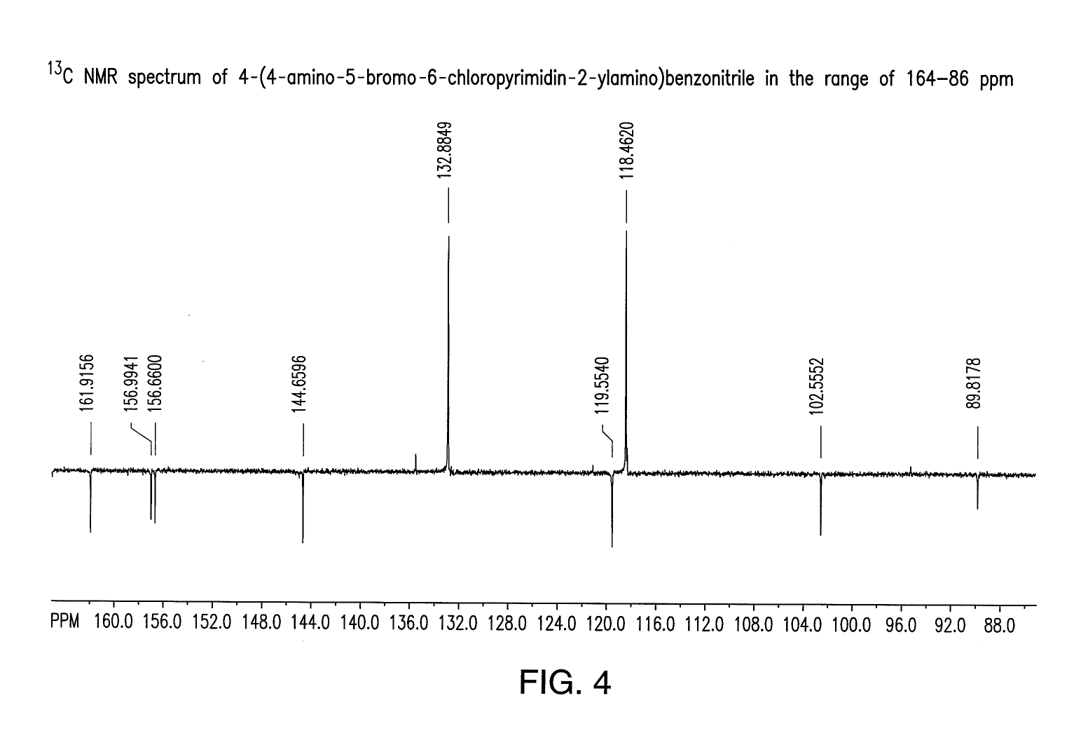

¹H NMR and ¹³C NMR spectra for this product are shown in FIGS. 1-2 and 3-4, respectively.

### Example 2b

### Preparation of 4-(4-amino-5-bromo-6-chloropyrimidin-2-ylamino)benzonitrile (“Compound 3a”)

A suspension of 4-(4-amino-6-chloropyrimidin-2-ylamino)benzonitrile (“CAPBN”, compound 1a; 2.0 g; 8.14 mmol) in methanol (100 ml) was cooled at 0-5° C. Bromine (439.2 μl; 8.55 mmol; 1.05 eq) was added dropwise to the suspension over 10 minutes. The resulting reaction mixture was stirred at 0-5° C. for 30 minutes, and then water (4.15 ml) and 10% aqueous NaOH (3.51 ml; 9.74 mmol; 1.2 eq) was added dropwise. The suspension was stirred 1 h at 0-5° C. A solid was separated from the suspension by filtration and washed with a MeOH/water mixture (1:1, 10 ml), and dried (5 h/40° C./10 mbar).
Yield: 2.02 g (76.5%) of 4-(4-amino-5-bromo-6-chloropyrimidin-2-ylamino)benzonitrile.
Purity (HPLC/MS): 90 Area %
M+326.4 (±2)

### Example 3a

### Preparation of 4-(6-amino-5-bromo-2-(4-cyanophenylamino)pyrimidin-4-yloxy)-3,5-dimethylbenzonitrile (Etravirine)

To a solution of 4-hydroxy-3,5-dimethylbenzonitrile (“DMHB”; 181.3 mg; 1.23 mmol; 1 eq) in N-methylpyrrolidone (“NMP”; 5 ml) cooled at 0-5° C., was added sodium hydride (60% suspension in mineral oil; 59.1 mg; 1.48 mmol; 1.2 eq) under argon. The resulting suspension was stirred 10 minutes at 0-5° C., and then 4-(4-amino-5-bromo-6-chloropyrimidin-2-ylamino)benzonitrile (“BCAPBN”) (400 mg; 1.23 mmol; 1 eq) was added in one portion. The reaction mixture was stirred: 30 minutes at 0-5° C., 16 hours at room temperature, and an additional 6 hours at 120-142° C. The resulting mixture was then cooled at 0-5° C., and water (5 ml) was added dropwise, and the resulting mixture was stirred 1.5 hours at 0-5° C. A solid was separated from the suspension by filtration and dried (4h/40° C./10 mbar and r.t./10 mbar/overnight).
Yield: 442 mg (82.5%) of 4-(6-amino-5-bromo-2-(4-cyanophenylamino)pyrimidin-4-yloxy)-3,5-dimethylbenzonitrile.
Purity (HPLC/MS): 93.8 Area %.
M+435.7

### Example 3b

### Preparation of 4-(6-amino-5-bromo-2-(4-cyanophenylamino)pyrimidin-4-yloxy)-3,5-dimethylbenzonitrile (Etravirine)

To a solution of 4-hydroxy-3,5-dimethylbenzonitrile (“DMHB”; 181.3 mg; 1.23 mmol; 1 eq) in N-methylpyrrolidone (“NMP”; 5 ml) cooled at 0-5° C. under argon, was added sodium hydride (60% suspension in mineral oil; 59.1 mg; 1.48 mmol; 1.2 eq). The resulting suspension was stirred 10 minutes at 0-5° C., and then 4-(4-amino-5-bromo-6-chloropyrimidin-2-ylamino)benzonitrile (“BCAPBN” Compound 3a) (400 mg; 1.23 mmol; 1 eq) was added in one portion. The reaction mixture was stirred: 30 minutes at 0-5° C., 16 hours at room temperature and 25 hours at 120° C. The resulting mixture was cooled at 0-5° C., and water (7 ml) was added dropwise, and this mixture was stirred 1.5 hours at 0-5° C. A solid was separated from the suspension by filtration and dried (5 h/40° C./10 mbar and 65 h/r.t./10 mbar).
Yield: 505 mg (94%) of 4-(6-amino-5-bromo-2-(4-cyanophenylamino)pyrimidin-4-yloxy)-3,5-dimethylbenzonitrile.
Purity (HPLC/MS): 92.8 Area %.
M+435.7

### Example 3c

### Preparation of 4-(6-amino-5-bromo-2-(4-cyanophenylamino)pyrimidin-4-yloxy)-3,5-dimethylbenzonitrile (Etravirine)

To a solution of 4-hydroxy-3,5-dimethylbenzonitrile (“DMHB”; 181.3 mg; 1.23 mmol; 1 eq) in NMP (5 ml), cooled at 0-5° C. under argon, was added K₂CO₃ (340.5 mg; 2.46 mmol; 2 eq) and KI (51.1 mg; 0.31 mmol; 25 mol %). The resulting suspension was stirred 10 minutes at 0-5° C., and then 4-(4-amino-5-bromo-6-chloropyrimidin-2-ylamino)benzonitrile (“BCAPBN” Compound 3a) (400 mg; 1.23 mmol; 1 eq) was added in one portion. The reaction mixture was stirred: 30 minutes at 10° C. and 24 hours at 125° C. The resulting mixture was cooled at 0-5° C., and water (7 ml) was added dropwise, and this mixture was stirred 1.5 hours at 0-5° C. A solid was separated from the suspension by filtration and dried (5 h/40° C./10 mbar and 65 h/r.t./10 mbar).
Yield: 462 mg (86.2%) of 4-(6-amino-5-bromo-2-(4-cyanophenylamino)pyrimidin-4-yloxy)-3,5-dimethylbenzonitrile.
Purity (HPLC/MS): 93.8 Area %.
M+435.7

### Example 3d

### Preparation of 4-(6-amino-5-bromo-2-(4-cyanophenylamino)pyrimidin-4-yloxy)-3,5-dimethylbenzonitrile (Etravirine)

To the solution of 4-hydroxy-3,5-dimethylbenzonitrile (DMHB; 226.6 mg; 1.54 mmol; 1 eq) in NMP (5 ml), cooled at 0-5° C., K₂CO₃ (851.6 mg; 6.16 mmol; 4 eq) and KI (63.9 mg; 0.358 mmol; 25 mol %) was added under N₂. The resulting suspension was stirred 10 minutes at 0-5° C. and then 4-(4-amino-5-bromo-6-chloropyrimidin-2-ylamino)benzonitrile (“BCAPBN”, Compound 3a) (500 mg; 1.54 mmol; 1 eq) was added in one portion. The reaction mixture was stirred: 30 minutes at 10° C. and 26 h at 120° C. The resulting mixture was cooled to 0-5° C., and water (7 ml) was added dropwise and this mixture was stirred 1.5 h/0-5° C. A solid was separated from the suspension by filtration and dried (4 h/40° C./10 mbar and overnight/r.t./10 mbar).
Yield: 597 mg (89%) of 4-(6-amino-5-bromo-2-(4-cyanophenylamino)pyrimidin-4-yloxy)-3,5-dimethylbenzonitrile.
Purity (HPLC/MS): 95.7 Area %.
M+435.7

### Example 4a

### Purification of 4-(6-amino-5-bromo-2-(4-cyanophenylamino)pyrimidin-4-yloxy)-3,5-dimethylbenzonitrile (Etravirine)

4-(6-Amino-5-bromo-2-(4-cyanophenylamino)pyrimidin-4-yloxy)-3,5-dimethyl-benzonitrile (etravirine crude), was suspended in acetone (14.6 ml), and a solution of p-toluenesulfonic acid monohydrate (280 mg) in acetone (2.9 ml) was added dropwise. The resulting mixture was stirred at room temperature for 1 hour. The suspension was then filtered and the collected solid was washed with 2.3 ml of water.

The thus obtained wet etravirine tosylate was suspended in acetone (14.6 ml) and 10% NaOH (0.578 ml) was added dropwise. The mixture was warmed to 40-45° C. and water (7.6 ml) was added. The resulting suspension immediately dissolved and then quickly precipitated afterwards. The obtained suspension was then cooled to 0-5° C., stirred for 1 hour and filtered. The collected solid was washed with water (2×1.2 ml) and dried (4 h/50° C./10 mbar).
Yield: 350 mg (70%) of 4-(6-amino-5-bromo-2-(4-cyanophenylamino)pyrimidin-4-yloxy)-3,5-dimethylbenzonitrile.
Purity (HPLC/MS): 100 Area %.
M+435.7

### Example 4b

### Purification of 4-(6-amino-5-bromo-2-(4-cyanophenylamino)pyrimidin-4-yloxy)-3,5-dimethylbenzonitrile (Etravirine)

4-(6-Amino-5-bromo-2-(4-cyanophenylamino)pyrimidin-4-yloxy)-3,5-dimethylbenzo-nitrile (etravirine crude, 4.8 g) was suspended in acetone (120 ml) and a solution of p-toluene-sulfonic acid monohydrate (2.3 g, 12.09 mmol, 1.1 eq) in acetone (24 ml) was added dropwise. The resulting mixture was stirred at r.t. 1 h. The suspension was filtered and the collected solid was washed with acetone (20 ml).

The thus obtained wet etravirine tosylate was suspended in acetone (120 ml) and 10% NaOH (4.7 ml, 13.05 mmol, 1.2 eq) was added dropwise. The mixture was heated to 40° C. and water was added (62 ml). The resulting suspension immediately dissolved and quickly precipitated afterwards. This suspension was cooled to 0-5° C., stirred for 1 h and filtered. The obtained solid was washed with water (2×9.5 ml) and dried (5 h/40+2 h/50° C./10 mbar).
Yield: 3.8 mg (79.2%) of 4-(6-amino-5-bromo-2-(4-cyanophenylamino)pyrimidin-4-yloxy)-3,5-dimethylbenzonitrile. Purity (HPLC/MS): 99.6 Area %.

### Example 5

### Preparation of 4-(4-amino-6-chloropyrimidin-2-ylamino)benzonitrile (“Compound 1a”)

To a solution of 4-aminobenzonitrile (ABN; 7 g; 59.3 mmol) in 2-propanol (110 ml), 2,6-dichloropyrimidin-4-amine (“DCAP”; 10.2 g; 62.2 mmol) was added at room temperature. Hydrochloric acid in 2-propanol solution (15.4%, 72 g, 313 mmol of HCl) was added dropwise. The resulting suspension was heated to reflux (84° C.) and stirred for 24 hours. The suspension was then cooled to 20-25° C. and stirred for 1 hour at 20-25° C. A solid was separated from the suspension by filtration and washed with 2-propanol (20 ml) and dried (2 h/40° C./10 mbar).

Yield: 11.88 g (71.6%) of 4-(4-amino-6-chloropyrimidin-2-ylamino)benzonitrile hydrochloride. Purity (HPLC/MS): 98.2 Area %

### Example 6

### Preparation of 4-(4-amino-6-chloropyrimidin-2-ylamino)benzonitrile (“Compound 1a”)

To a solution of 4-aminobenzonitrile (ABN; 7 g; 59.3 mmol) in 2-propanol (190 ml), 2,6-dichloropyrimidin-4-amine (“DCAP”; 10.2 g; 62.2 mmol) was added at room temperature. The resulting suspension was heated to reflux (84° C.) and stirred for 24 hours. The suspension was then cooled to 20-25° C. and stirred for 1 hour at 20-25° C. A solid was separated from the suspension by filtration and washed with 2-propanol (20 ml) and dried (2 h/40° C./10 mbar).

Yield: 12.54 g (75.0%) of 4-(4-amino-6-chloropyrimidin-2-ylamino)benzonitrile hydrochloride.

Purity (HPLC/MS): 98.3 Area %

### Example 7

### Preparation of 4-(4-amino-6-chloropyrimidin-2-ylamino)benzonitrile (“Compound 1a”)

To a solution of 4-aminobenzonitrile (ABN; 7 g; 59.3 mmol) in 2-butanol (190 ml), 2,6-dichloropyrimidin-4-amine (“DCAP”; 10.2 g; 62.2 mmol) was added at room temperature. The resulting suspension was heated to reflux (102° C.) and stirred for 20 hours. The suspension was then cooled to 20-25° C. and stirred for 1 hour at 20-25° C. A solid was separated from the suspension by filtration and washed with 2-butanol (20 ml) and dried (2 h/50° C./10 mbar).

Yield: 12.20 g (73.0%) of 4-(4-amino-6-chloropyrimidin-2-ylamino)benzonitrile hydrochloride.

Purity (HPLC/MS): 99.0 Area %

### Example 8

### Preparation of 4-(4-amino-6-chloropyrimidin-2-ylamino)benzonitrile (“Compound 1a”)

To a suspension of 4-aminobenzonitrile (ABN; 0.50 g; 4.23 mmol) in a solvent such as ethanol, water, 2propanol, or 2-butanol (10 mL; concentration of ABN is 50 g/L), 2,6-dichloropyrimidin-4-amine (DCAP; 0.83 g; 5.08 mmol; 1.2 mol Eq) was added at room temperature. An acid, such as 36% HCl, 85% H₃PO₄, 65% HNO₃, 96% H₂SO₄, 47% HBr, p-TsOH, CCl₃COOH, (3.60 molEq) was added at room temperature and the reaction mixture was warmed up to 80° C. and stirred for 20 h.

The reaction mixture was then cooled to room temperature, filtered and the collected solid as washed efficiently with the solvent used in the synthesis; and dried for 6 h at 40° C. and 1 mbar. Yields were calculated based on the acid used, and on the isolated mass.

### Example 9

### Preparation of 4-[(4-amino-5-bromo-6-chloropyrimidin-2-yl)amino]benzonitrile (“Compound 3a”)

400 ml of 2-butanol and 31.4 g (111 mmol) of 4-[(4-amino-6-chloropyrimidin-2-yl)amino]benzonitrile hydrochloride were mixed for 15 minutes. Bromine (36 g, 225 mmol) was then added dropwise during 45 minutes while maintaining the temperature of the reaction mixture at 20-25° C. The suspension was mixed for 30 minutes and the pH was set to 9.1 by addition of NaOH (40%, 42 ml). The obtained suspension was then heated to 40° C., mixed for 60 minutes and cooled to 20-25° C. and further mixed for 90 minutes. The obtained crystals were filtrated over a black ribbon under vacuum and washed with 50 ml of 2-butanol, 50 ml of water, and again with 50 ml of 2-butanol. Then, they were dried at 50° C. in a vacuum oven.

Yield: 35.95g (99.5%) of 4-[(4-amino-5-bromo-6-chloropyrimidin-2-yl)amino]benzonitrile Purity (HPLC/MS): 97.3 Area %

1. A compound of Formula 1 of the following structure: 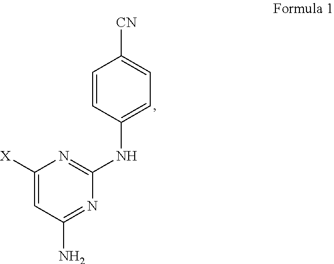
or a salt thereof, wherein X is a halogen or a tosyl group.

2. The compound according to claim 1, wherein X is halogen.

3. The compound according to claim 1, wherein the compound is in isolated form.

4. The compound according to claim 1, wherein the compound is in crystalline form.

5. A process for preparing a compound of Formula 1 according to claim 1, or a salt thereof, said process comprising reacting a compound of Formula 2 or a salt thereof of the following structure: 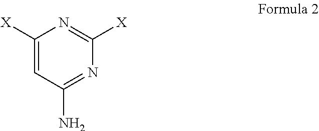
with 4-aminobenzonitrile, wherein X is a halogen or a tosyl group.

6. The process according to claim 5, wherein X is —Cl and the compound prepared is compound 1a:

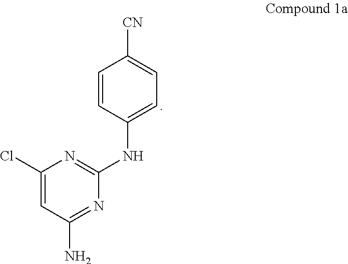 [SMILES: N#Cc1ccc(Nc2nc(N)cc(Cl)n2)cc1]

7. (canceled)

8. (canceled)

9. A process for preparing a compound of Formula 3 or a salt thereof: 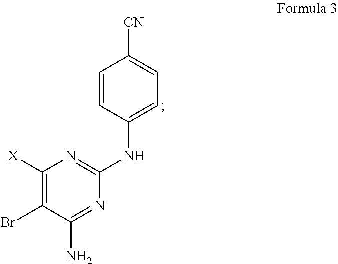
comprising providing a compound of Formula 1 or a salt thereof: 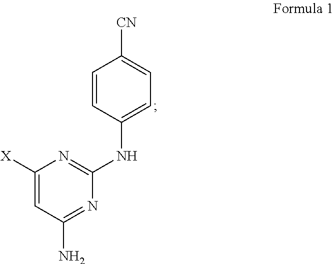
and brominating said compound to produce the compound of Formula 3, or a salt thereof; wherein X is a halogen or a tosyl group.

10. The process of claim 9, wherein X is halogen.

11. (canceled)

12. The process of claim 9 further comprising converting the compound of Formula 3 or a salt thereof to Etravirine or a salt thereof.

13. (canceled)

14. A process for preparing Etravirine or a salt thereof, said process comprising providing a compound of Formula 1 or a salt thereof according to claim 1: 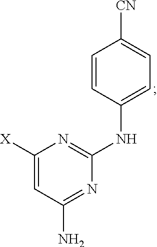
and converting the compound of Formula 1 or a salt thereof to Etravirine or a salt thereof, wherein X is a halogen or a tosyl group.

15. The process of claim 14 wherein X is halogen.

16. The process of claim 14, wherein the converting step comprises:
a. brominating a compound of Formula 1 or a salt thereof to produce a compound of Formula 3 or a salt thereof: 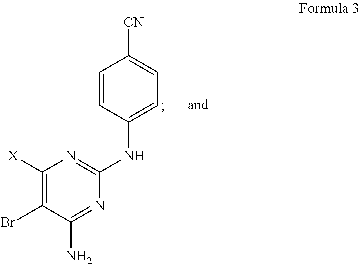
b. converting the obtained compound of Formula 3 or a salt thereof to Etravirine or a salt thereof.

17. (canceled)

18. The process of claim 16, wherein the converting in step b) comprises reacting the compound of Formula 3 or a salt thereof and 4-hydroxy-3,5-dimethylbenzonitrile (“DMHB”) of the following structure:

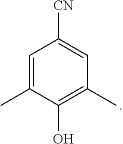

19. A process for preparing Etravirine or a salt thereof comprising:
a. reacting a compound of Formula 2 or a salt thereof: 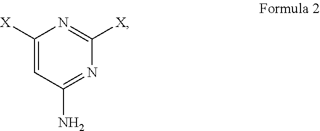
and 4-aminobenzonitrile (“ABN”) to obtain a compound of Formula 1 or a salt thereof: 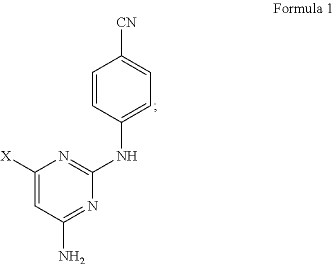
b. brominating the compound of Formula 1 or a salt thereof to obtain a compound of Formula 3 or a salt thereof: 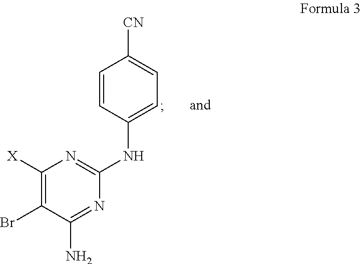
c. reacting the compound of Formula 3 or a salt thereof and 4-hydroxy-3,5-dimethylbenzonitrile (“DMHB”) to obtain Etravirine or a salt thereof; wherein X is a halogen or a tosyl group.

20. The compound according to claim 2, wherein X is Cl.

21. The process according to claim 10, wherein X is Cl.

22. The process according to claim 15, wherein X is Cl.
